(19) United States
(12) Patent Application Publication
(10) Pub. No.: US 2014/0276300 A1
(43) Pub. Date: Sep. 18, 2014

(54) ORTHOPEDIC SLEEVE AND METHOD FOR PRODUCING THE SAME

(71) Applicant: OTTO BOCK HEALTHCARE GMBH, Duderstadt (DE)

(72) Inventors: Holger Reinhardt, Kempen (DE); Matthias Vollbrecht, Herzberg (DE)

(73) Assignee: OTTO BOCK HEALTHCARE GMBH, Duderstadt (DE)

(21) Appl. No.: 14/356,353

(22) PCT Filed: Nov. 16, 2012
(86) PCT No.: PCT/EP2012/004774

§ 371 Date: May 5, 2014

(30) Foreign Application Priority Data
Nov. 16, 2011 (DE) 10 2011 118 617.8

(51) Int. Cl. A61F 5/01

(52) U.S. Cl. CPC A61F 5/0104
USPC 602/5; 156/245

(57) ABSTRACT

The invention relates to an orthopedic sleeve (1) for placing against a body, having a flexible basic body (10) with at least one plastics-material stabilizing element (20) arranged thereon, wherein the stabilizing element (20) has regions (21, 22, 31, 32) which have a cross section reduced in relation to the rest of the cross section and in which the stabilizing element (20) is adhesively bonded to the basic body (10) and/or connected integrally to the basic body (10). The invention also relates to a method for producing such a sleeve.

The invention relates to an orthopedic sleeve for placing on a body, having a flexible basic body with at least one stabilizing element made of plastic arranged thereon, and to a method for producing such a sleeve. The sleeve is designed in particular as a liner as interface between a stump and a prosthesis socket, but it can also be designed as a bandage, which is applied to the body for cushioning and/or support.

Bandages, in particular joint bandages, are applied for supporting or for protecting the muscles and ligaments. For this purpose, the bandage is pulled over or wound around the joint that is to be protected and is then optionally fixed. In contrast to immobilizing orthoses, bandages allow the limbs connected via the joint to move relative to each other to a certain extent, in at least one degree of freedom. For this purpose, the bandages have a basic body made of a flexible material, for example a foam or a textile. To support the stabilization effect, stabilizing elements are arranged on the basic body and have greater strength and stiffness than the basic body, such that, in addition to protection against mechanical influences, increased restriction on movement is obtained.

Moreover, so-called liners are known which are fitted on a stump of a limb in order to produce an interface to a prosthesis. The liners can be designed as plastics liners or silicone liners and can be made airtight or coated with adhesive on the inside. By means of a partial vacuum, the liner holds a prosthesis socket on the stump, which partial vacuum can be applied between the liner and the prosthesis socket. Alternatively, the liner has, on the outside thereof, locking elements via which the prosthesis socket is held on the liner. By virtue of the nature of its inner surface, the liner adheres to the stump.

US 2008/0039757 A1 describes a flexible bandage with a framework having a surface for placing on an area of a body, wherein the framework has a multiplicity of permanent openings. A woven fabric or foam can be applied to the framework, for example by welding, adhesive bonding or sewing.

EP 876 130 B1 describes an orthopedic brace with a retainer made of a one-piece injection-molded component having regions of different thicknesses, in order to adapt to the contour of a selected part of a limb. A layer of elastic material is mounted inside the retainer, in order to cushion the retainer when it engages on the selected part of the limb. The retainer can be connected to the cushioning by adhesive bonding or in some other way.

U.S. Pat. No. 6,024,712 describes an ankle-joint orthosis with an inner textile part extending at least partially around the ankle joint. An outer brace is injection-molded into the textile and supplies additional support for the joint against unintended movements.

CA 2,398,059 A1 describes an orthopedic brace with a flexible inner part and an outer brace which is molded directly onto the flexible inner part.

A problem is that the direct binding of the cushioning material to the stabilizing elements leads to abrupt stresses, which is a disadvantage in terms of durability and wearing comfort.

The object of the present invention is to make available an orthopedic sleeve in which a firm and lasting union is achieved between the basic body and the stabilizing element.

According to the invention, this object is achieved by a sleeve having the features of the main claim, and by a method having the features of the additional independent claim. Advantageous embodiments and refinements of the invention are set forth in the dependent claims, the description and the figures.

The orthopedic sleeve, which is provided for placing on a body and is composed of a flexible basic body with at least one stabilizing element made of plastic arranged thereon, is characterized in that the stabilizing element has regions which have a cross section reduced in relation to the rest of the cross section, and in which the stabilizing element is adhesively bonded to the basic body and/or connected cohesively to the basic body. By providing the connection in the regions having a reduced cross section, it is possible to make available connection regions with sufficient elasticity, such that the risk of the basic body tearing or coming loose from the stabilizing element is reduced. At least a transition is thus made available by a cross-sectional reduction, such that the edge regions of the stabilizing element have, by comparison with the rest of the cross section, an increased flexibility, which reduces the risk of the connection to the basic body being torn out or similar. The sleeve can be part of an orthosis or prosthesis and can additionally be incorporated in a system of orthotic or prosthetic components. The sleeve can likewise exert an independent supporting and protecting function when worn as a bandage.

In a refinement of the invention, provision is made that the stabilizing element is applied to a flexible substrate material. This application can be carried out by injection-molding the stabilizing element onto or from behind the substrate material. The substrate material then forms those regions that are connected cohesively to the basic body and/or adhesively bonded thereto. By the interposition of the substrate material when securing the stabilizing element or the stabilizing elements on the basic body, it is possible to bind the stabilizing elements to the substrate material in a very durable manner and then connect the latter in turn to the basic body, for example by welding, adhesive bonding or the like. With a stabilizing element injection-molded onto the substrate material in this way, a high level of reproducibility of the connection is possible, and the flexible substrate material is also able to allow a mobility, however slight, of the stabilizing element relative to the basic body, as a result of which tears at transitions from the stabilizing element to the substrate material and from the substrate material to the basic body can be avoided. It is also possible to produce articulated connections by injection-molding onto or from behind the substrate material, for example by two stabilizing elements being arranged lying opposite each other such that they form a hinge.

Advantageously, the substrate material protrudes, at least in part, beyond the periphery of the stabilizing element, such that regions are formed that are suitable for adhesively bonding or welding to the basic body. By this protrusion beyond the stabilizing elements, the contact surface is also enlarged, so that stable securing of the stabilizing element to the basic body is ensured.

In a refinement of the invention, provision is made that adhesive bonding or cohesive connection to the basic body is effected only in the region or regions of reduced thickness protruding beyond the periphery of the stabilizing elements, thereby ensuring sufficient elasticity and binding to the basic body.

On the side directed toward the basic body in the assembled state, the substrate material can be laminated with a plastic, so as to provide better connectability to the material of the basic body. The laminated-on plastic and the basic body are advantageously a pairing of materials that can be efficiently welded or adhesively bonded to each other. By means of the plastic that is laminated on, it is possible that the substrate material does not need to be optimized in respect of a connection to the basic body, and instead the connection to the stabilizing element can be optimized, for example, while the plastic that is laminated on produces the connection to the material of the basic body. By application of a plastic, two materials are thus interposed between the stabilizing element and the basic body, such that the different mechanical requirements of the stabilizing element and of the basic body can be more easily met. The plastic that is laminated on is advantageously different than the substrate material and can be polyurethane. It is likewise possible for the plastic that is laminated on to be configured as a dipole material.

The substrate material can be designed as a textile, in particular as a velour, particularly advantageously here a polyamide velour, since the material of the stabilizing element and also that of the laminated-on plastic anchors itself very efficiently in the velour surface and, in this way, a good adherence and connection can be achieved between the stabilizing element and the substrate material, likewise between the laminated plastic and the substrate material.

The stabilizing element can be made of a material that has a lower melting point than the material of the basic body or of the substrate material. It is thereby possible for the stabilizing element to be injection-molded onto the substrate element or onto the basic body without the substrate material or the basic body being deformed by the heat input, or without the material properties of the substrate material or of the basic body changing. The stabilizing element can be made of polyurethane or polyethylene, for example.

In a refinement of the invention, provision is made that several stabilizing elements, connected to each other in an articulated manner, are secured on the basic body. The securing can take place either directly in the thinner regions or via the substrate element. For this purpose, the stabilizing elements are designed such that they establish a form-fit connection to each other, for example by one stabilizing element being designed as an eyelet into which a pin of the second stabilizing element engages. The assembly of the two stabilizing elements to form a joint is advantageously carried out prior to the securing of the substrate material on the basic body, although it can also be carried out subsequently, when the two stabilizing elements are correspondingly positioned and secured on the basic body.

The basic body is advantageously designed as a textile, in particular as an elastic textile, for example as an elastic knit or elastic woven fabric. The textile can be coated on the inner face and/or the outer face in order to adjust properties of the sleeve, for example of the liner or of the bandage, in the desired manner. Thus, the inner face and outer face can have an airtight and/or adhering coating, in order to achieve good contact on the body part or on a stump. Alternatively, the basic body can also be made from foam, for example neoprene. It is also possible for the sleeve to be made wholly or partially of plastic or silicone, particularly in the embodiment of the sleeve as a liner.

To permit good adherence of the stabilizing element, the substrate material is connected to the stabilizing element over the entire surface, and the substrate material can likewise be connected to the basic body over the entire surface, which represents an alternative to securing only at the protruding regions or at the narrowed regions of the stabilizing element.

In one embodiment of the invention, provision is made that the sleeve is designed as a liner, wherein the substrate material has an adhesive, in particular airtight coating. This coating can be applied on the inner face and/or the outer face. The stabilizing element can be designed as a distal cap or part of another supporting device or securing device for securing or receiving a prosthesis socket. The liner can have a closed cross section. Alternatively, the sleeve is designed as a bandage for placing on the body. The bandage can have a closed cross section and can be pulled over a limb. It is likewise possible that the sleeve is designed as a bandage with an open cross section and, after being applied, is closed or is fixed in the desired position.

The method for producing a sleeve as claimed in one of the preceding claims comprises the steps whereby a stabilizing element made of a plastic is injection-molded onto or from behind a flexible substrate material, and the substrate material is then welded and/or adhesively bonded to a flexible basic-body blank. The basic body or the basic body blank can be closed or also open, for example as a closeable lumbar orthosis, or as a closed knee bandage which is fitted by being pulled on over the lower leg. In addition to a design in which the sleeve is open at both ends and in which a substantially tubular basic body is present, it is also possible to provide a sleeve which is open at one end and which, for example, extends over a stump and can be arranged between the limb and a prosthetic device.

Several stabilizing elements can be injection-molded onto a substrate material and then separated from the substrate material so as to form at least one region protruding beyond the periphery of the stabilizing element. It is thereby possible to produce the stabilizing elements separately in an automated manner and, when necessary, to cut the stabilizing elements out from a basic material and then connect them to the basic body.

The side of the substrate material directed away from the stabilizing element can be laminated with a plastic, in particular polyurethane, prior to the welding or adhesive bonding, in order to be able to achieve a better connection between the substrate material and the basic body. The plastic that is laminated on advantageously has thermoplastic properties, so as to provide a sufficient elasticity even after a thermal connection.

The substrate material can be connected to the basic-body blank by means of high-frequency welding, as a result of which it is possible to achieve increased variability of the weld width by suitable guiding of the electrode. A narrow or wide connection of the substrate material to the basic body can then be achieved depending on the charge.

The substrate material can be connected to the basic-body blank only in a region protruding beyond the stabilizing element, such that an increased elasticity is achieved and a direct connection of the stabilizing element to the basic body is avoided.

Illustrative embodiments of the invention are explained in more detail below with reference to the attached figures, in which:

FIG. 1 shows a cross-sectional view of a basic-body blank, with a stabilizing element welded thereon;

FIG. 2 shows a cross-sectional view of a stabilizing element on a substrate material;

FIG. 3 shows a cross-sectional view of a stabilizing element with substrate material mounted on a basic body;

FIG. 4 shows a variant of FIG. 3;

FIG. 5 shows a knee bandage with an applied stabilizing element;

FIG. 6 shows a variant of FIG. 5;

FIG. 7 shows a back orthosis with applied stabilizing elements;

FIG. 8 shows an ankle-joint orthosis with applied stabilizing elements;

FIG. 9 shows a knee bandage with several stabilizing elements connected in an articulated manner;

FIG. 10 shows a perspective view of a knee orthosis;

FIG. 11 shows a knee orthosis according to FIG. 9 from another angle;

FIG. 12 shows a cross-sectional view of the securing of stabilizing elements on a basic body; and

FIG. 13 shows a cross-sectional view of a sleeve in the form of a liner.

FIG. 1 shows a cross-sectional view through a conventional sleeve in the form of a bandage with a basic body 10 made of a plastic or textile, and with a stabilizing element 20 applied thereon. The basic body and the stabilizing element 20 are made of a material allowing them to be welded to each other. Arranged below the basic body 10 is a high-frequency electrode 40, by which it is possible to secure the stabilizing element 20 on the basic body 10. Securing seams 11, 12 along the contact faces of the electrode 40 on the basic body 10 are indicated by broken lines.

FIG. 2 shows a stabilizing element 20 which is connected, in particular welded, to a substrate material 30. The stabilizing element 20 can be made of any suitable plastic, for example of TPU, PVC or PA. In the illustrative embodiment shown, the substrate material 30 is designed as a plastics film, which can be made from TPU or PA. The stabilizing element 20 and the substrate material 30 are welded to each other, for example by the electrode 40. As an alternative to only partial welding, the stabilizing element 20 can be welded to the substrate material 30 over the entire surface. A plastics layer 35 can be laminated onto the surface of the substrate material 30 directed away from the stabilizing element 20 and can be made of a material different than the material of the substrate material 30. It is likewise possible for the stabilizing element 20 to be adhesively bonded to the substrate material 30, in which case it is also possible for the adhesive bonding to be effected over the entire surface or only partially.

As an alternative to a design of the substrate material 30 as a film, it can also be designed as a textile on which the stabilizing element 20 is secured. The stabilizing element can be secured on a textile substrate material 30 likewise by welding or adhesive bonding, or by being injection-molded onto or from behind the substrate material.

As an alternative to a subsequent application of the substrate material 30 to the stabilizing element 20, the production of a stabilizing element 20 with a substrate material arranged thereon can be carried out by two-component injection molding.

FIG. 3 shows how the stabilizing element 20 is applied together with the substrate material 30 to a basic body 10 or a basic-body blank. Laterally alongside the stabilizing element 20, two regions 31, of the substrate material 30 are provided which protrude beyond the periphery of the stabilizing element 20. Along these regions 31, 32, the substrate material 30 is welded to the basic body 10, which can be designed as a textile, for example as an elastic knit, or from foam, for example from neoprene. The weld seams 11, 12 are indicated by broken lines in the basic body 10.

As a variant to only partial welding or connection to the basic body 10, it is possible that the substrate material 30 can be connected to the basic body 10 over the entire surface, for example adhesively bonded or welded over the entire surface. Since the stabilizing element 20, which is made of plastic or at least has a layer of plastic on the underside for connection to the substrate material, is provided with the substrate material 30, it is possible to make available a coupling element which is adapted to the material of the basic body 10, such that a direct cohesive or adhesive connection of the generally stiff stabilizing element 20 to the generally flexible basic body 10 is avoided. The indirect coupling of the stabilizing element 20 to the basic body 10 reduces the risk of tears forming, and optimized material properties can be obtained for the basic body 10 and the stabilizing element 20, without having to take into consideration a fundamental connectability of the two components to each other. For example, the stabilizing element 20 can have a high flexural strength, while the design of the basic body 10 can be optimized in respect of wearability, skin compatibility and elasticity.

A variant of the invention is shown in FIG. 4 in which, instead of a two-part design of the stabilizing element 20 with the substrate material 30, a one-part stabilizing element 20 is provided which, seen in the longitudinal direction, has lateral regions 21, 22 which have a smaller cross section than the area of the stabilizing element 20 lying between them. The lateral regions 21, 22 permit an increased elasticity and mobility, with the result that there is only a slight stiffening of the basic body 10 in the connection region, particularly in the case of high-frequency welding via the electrodes 40 along the lateral regions 21, 22 and binding along the weld seams 11, 12. This reduces the risk of the stabilizing element 20 coming loose from the basic body 10 on account of their having different flexibilities.

FIG. 5 is a schematic representation of a closed bandage 1 with a substantially cylindrical, optionally pre-flexed basic body 10, on the outside of which a stabilizing element 20 is applied. The stabilizing element 20 corresponds to the stabilizing element 20 of FIG. 4 and is constructed in one part and secured on the basic body 10 along the thinner regions 21, 22. The securing can be done by adhesive bonding or welding.

A variant of the invention is shown in FIG. 6 in the form of a knee bandage. A stabilizing element 20 in the form of a plastics spring is arranged laterally on the outer face of the basic body 10, which has a closed design. The plastics spring is applied to a substrate material 30, which can be designed as a textile, in particular as a velour. It can be applied to the substrate material 30, for example, by being injection-molded onto or from behind the latter, by high-frequency welding or by adhesive bonding. By way of the substrate material 30, the plastics spring is then secured on the basic body 10, for example by adhesive bonding or high-frequency welding.

Additional components 36 can be provided on the substrate material, for example a velcro fastener or a tightening loop, which can be applied or formed during the production of the substrate material 30, if appropriate in conjunction with the production of the connection to the stabilizing element 20.

The stabilizing element 20 can be prefabricated together with the substrate material 30 in an automated manner, such that a series of stabilizing elements 20 can be applied at intervals from each other on a substrate-material blank. From this blank, with the stabilizing elements applied thereon, it is then possible, as necessary, to cut out a suitable stabilizing element or several stabilizing elements together with an excess length of substrate material.

FIG. 7 shows a variant of the invention, in which the sleeve 1 is designed as a back orthosis, having an open basic-body blank, at the ends of which there are connecting elements, for example in the form of a velcro fastener, via which the back orthosis can be closed. On the outer face, stabilizing elements 20 are secured at intervals from one another on the basic body 10 via the substrate material 30.

A further variant of the invention is shown in FIG. 8, which depicts an ankle-joint orthosis. On the outer face, a stabilizing element 20, which is secured on a substrate element 30, is arranged respectively on the medial and lateral aspects. As also in the other sleeves designed as bandages, the basic body can be made of a foam, for example neoprene, or an elastic knit. The substrate material 30 can be made of a velour, another textile or likewise a suitable plastic, for example a plastics film, or a foam. A fleeced area 28 or a velcro area can be arranged on the outer face of the stabilizing element 20, so as to be able to provide an additional stabilization via straps or the like.

A further variant of the invention is shown in FIG. 9, in which the sleeve 1 is designed as a knee orthosis. Here too, stabilizing elements 20 are secured via a substrate material 30 on the medial and lateral aspects of the outer face of the closed, tubular and pre-flexed basic body 10. The substrate material 30 can be connected to the basic body 10 partially or over the entire surface. On the side of the substrate material 30 directed away from the basic body 10, stabilizing elements 20 are arranged which are shaped such that they can form an articulated connection. The upper stabilizing element 20 has an annular recess into which a correspondingly shaped projection of the lower stabilizing element 20 engages, such that a hinge joint is obtained, which is preferably positioned in the area of the natural knee axis of the person wearing the orthosis. The substrate material 30 can have a loop area 38 on the outer face, for example of velour, on which a hook area of a strap can be secured.

The side of the substrate material 30 directed toward the basic body 10 can be laminated with a TPU film, which can be generated by indirect heat input, for example via high-frequency or low-frequency excitation, melted, and connected to the material of the basic body 10.

FIG. 10 shows a further variant of a sleeve 1 in the form of a knee orthosis with a flexible, elastic basic body 10 made of a textile, if appropriate a coated textile, with two laterally arranged stabilizing elements 20 which are connected to each other in an articulated manner. The articulated connection advantageously lies in the area of the natural joint axis and is advantageously positioned there. A recess 50 is worked into the front of the basic material 10 and is formed in the area of the patella, such that no pressure is exerted on the patella by the elastic material of the basic body 10. A support element 52, which exerts lateral pressure on the patella, in order to correct the position of the patella. The support element 52 is held by a first strap 56, which is secured in the area of the articulated connection of the stabilizing elements 20.

FIG. 11 shows the bandage 1 from the medial direction, with two second straps 54 secured on the support element 52 in order to fix the position in the medial direction.

FIG. 12 shows detailed views of cross sections through the stabilizing element 20 and the basic body 10. In the top view in FIG. 12, the stabilizing element 20 is in the form of a splint made of plastic, with regions 21, 22 which are formed laterally on the longitudinal extent of the stabilizing element 20 and along which the stabilizing element 20 is secured on the basic body 10. The securing is done, for example, by high-frequency welding or by adhesive bonding.

The bottom view in FIG. 12 shows a variant of the securing, in which a substrate material 30 is arranged between the stabilizing element 20 and the basic body 10 and has, laterally alongside the stabilizing element 20, regions 31, 32 in which the substrate material is welded to the basic body 10.

A variant of the invention is shown in FIG. 13, in which a sleeve 1 in the form of a liner is depicted in a cross-sectional view. The basic body 10 is composed of a substantially tubular element made of plastic and having a closed cross section. A closed distal portion of the basic body 10 is provided opposite a proximal opening. The basic body 10 is made of an airtight plastic or silicone material and receives a stump of a limb. A liner cap is arranged as stabilizing element 20 at the distal end of the basic body 10. Arranged inside this stabilizing element 20 is an insert 58, into which securing means can be screwed to fasten to a prosthesis socket, for example. Moreover, the stabilizing element has a flexible substrate material 30, which is secured on the liner cap. The substrate material 30 protrudes laterally beyond the stabilizing element 20 and extends in the proximal direction toward the opening of the basic body 10, such that the distal end of the basic body 10 is completely enclosed by the substrate material 30. The regions 31, 32 extending laterally to the proximal end are connected cohesively to the basic body 10, for example welded on or adhesively bonded.

1. An orthopedic sleeve for placing on a body, the orthopedic sleeve comprising:
a flexible basic body;
at least one stabilizing element made of plastic and comprising regions having a cross section reduced in relation to a cross section of a remainder of the stabilizing element, the stabilizing element being at least one of adhesively bonded to the basic body and connected cohesively to the basic body.

2. The orthopedic sleeve as claimed in claim 1, wherein the stabilizing element is applied to a flexible substrate material by being injection-molded onto or from behind the substrate material, and the substrate material forms the regions which are at least one of connected cohesively to the basic body and adhesively bonded to the basic body.

3. The orthopedic sleeve as claimed in claim 2, wherein the substrate material protrudes, at least in part, beyond a periphery of the stabilizing element.

4. The orthopedic sleeve as claimed in claim 1, wherein the at least one of adhesive bonding and cohesive connection to the basic body is effected only in the regions protruding beyond a periphery of the stabilizing element.

5. The orthopedic sleeve as claimed in claim 2, wherein the substrate material, on a side directed toward the basic body, is laminated with a plastic material.

6. The orthopedic sleeve as claimed in claim 5, wherein the laminated-on plastic and the basic body are designed such that they can be welded to each other or adhesively bonded to each other.

7. The orthopedic sleeve as claimed in claim 5, wherein the laminated-on plastic is different than the substrate material and comprises a polyurethane material.

8. The orthopedic sleeve as claimed in claim 2, wherein the substrate material is designed as a textile comprising a polyamide velour.

9. The orthopedic sleeve as claimed in claim 2, wherein the stabilizing element comprises a material that has a lower melting point than the basic body or the substrate material.

10. The orthopedic sleeve as claimed in claim 1, wherein the stabilizing element comprises polyurethane or polyethylene.

11. The orthopedic sleeve as claimed in claim 1, wherein several stabilizing elements, connected to each other in an articulated manner, are secured on the basic body.

12. The orthopedic sleeve as claimed in claim 1, wherein the basic body is designed from textile comprising an elastic knit or elastic woven fabric, from plastic, silicone, or from foam.

13. The orthopedic sleeve as claimed in claim 2, wherein the substrate material is connected over an entire surface to the stabilizing element.

14. The orthopedic sleeve as claimed in claim 1, wherein the orthopedic sleeve is designed as a liner, the substrate material has an adhering that comprises an airtight coating, the stabilizing element is designed as a distal cap, and the basic body is designed as a bandage.

15. A method for producing an orthopedic sleeve according to claim 1, the method comprising:
injection molding a plastic stabilizing element onto or from behind a flexible substrate material;
welding or adhesively bonding the substrate material to a flexible basic-body blank.

16. The method as claimed in claim 15, wherein several stabilizing elements are injection-molded onto a substrate material and then separated from the substrate material to form at least one region protruding beyond a periphery of the stabilizing element.

17. The method as claimed in claim 15, wherein a side of the substrate material directed away from the stabilizing element is laminated with a plastic comprising polyurethane, prior to the welding or adhesive bonding.

18. The method as claimed in claim 15, wherein the substrate material is connected to the basic-body blank using high-frequency welding.

19. The method as claimed in claim 15, wherein the substrate material is connected to the basic body only on a region protruding beyond the stabilizing element.

20. An orthopedic sleeve for placing on a body, the orthopedic sleeve comprising:
a flexible basic body;
at least one plastic stabilizing element comprising regions having a reduced cross-section relative to cross-sections of remaining portions of the stabilizing element;
wherein the stabilizing element is at least one of adhesively bonded to the basic body and connected cohesively to the basic body.
